(19) United States
(12) Patent Application Publication
(10) Pub. No.: US 2024/0423825 A1
(43) Pub. Date: Dec. 26, 2024

(54) A DEVICE WITH SPECIAL HOLDING MECHANISM FOR USE BY MALES WITH INCONTINENCE

(71) Applicant: Ramkumar M K, Chennai (IN)

(72) Inventor: Ramkumar M K, Chennai (IN)

(21) Appl. No.: 18/563,981

(22) PCT Filed: Mar. 24, 2022
(86) PCT No.: PCT/IN2022/050292

§ 371 Date: Nov. 24, 2023

(30) Foreign Application Priority Data
May 24, 2021 (IN) 202141023184

(51) Int. Cl. A61F 5/453; A61F 5/44

(52) U.S. Cl. CPC A61F 5/453; A61F 5/4401; A61F 5/4405; A61F 5/4408

(57) ABSTRACT

A male incontinence device comprises a holding device, a pouch, and a bead. The holding device comprises two cylindrical tubes and a tubular sponge. The pouch comprises an open end and a closed end and extends from an outside surface of the cylindrical tubes to extend beyond the penis that is inserted within, wherein a closed end of the pouch has a flared section that accommodates an absorbent unit to absorb urine. The bead is positioned at the open end of the pouch and integrated between the cylindrical tubes.

### FIELD OF INVENTION

The present invention relates to a device for collecting urine in solid form for convenience of male users with incontinence.

### BACKGROUND OF THE INVENTION

In the last decade or more there has been a perceptible shift in the ageing population. Medical advancement has increased longevity, but some problems related to lifestyle issues have increased. One of the prime concerns is frequent urination among the old.

In the current art, a diaper collects urine through several layers of perforated membranes to ultimately reach an absorbent layer where the urine is absorbed. There are several disadvantages with such diapers, however this is the most widely used product for adult incontinence due to lack of better alternatives. Over 70% people adopt this method even though the adult diaper is bulky and uncomfortable to wear particularly in tropical weather. There is a need for an improved product in this category.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The device disclosed here addresses the above-mentioned need for an improved device for male users suffering from incontinence. The male incontinence device comprises of 3 components which are holding mechanism, pouch, and sponge in the pouch, impregnated with absorbent. In an embodiment, the absorbent unit present at the closed end of the pouch is referred to a sponge material that has liquid absorbing properties, impregnated with an absorbent material that converts urine in liquid form to a solidified/gel form. The tubular sponge expands and contracts inversely to a varying diameter of one of the flaccid, firm and inflated state of the penis of user at different times.

The pouch has an open end with a bead and a closed end and cylindrical in shape, for example, like a condom. The holding device comprises two cylindrical tubes and a tubular sponge. The tubular sponge forms the inner most layer and is flush with the inner wall of the inner tube, and the tubular sponge ensures no back-flow of urine. The tubular sponge is in an expanded and squeezed position depending on state of the penis, wherein when the penis is flaccid, the tubular sponge is expanded, and when penis is turgid, the tubular sponge is squeezed. The sponge includes an absorbent powder like sodium polyacrylate.

Further, the pouch comprises an open end and a closed end and the pouch extends from the outside surface of the cylindrical tube to extend beyond the penis that is inserted within, wherein the closed end of the pouch has a flared section that accommodates an absorbent unit to absorb urine. The bead is present at the open end of the pouch and is secured between the cylindrical tubes.

In yet another embodiment, the cylindrical tubes that are connected to the pouch which covers the penis is held firm at the penis by two rods connected to the cylindrical tubes. In an embodiment, the absorbent unit expands into the flared closed end and is locked into position by a narrow neck of the pouch connecting the closed flared end with body of the pouch. In an embodiment, the cylindrical tubes comprise a flange that is integrated to the cylindrical tubes, where the flange comprises anchor holes into which an elastic thread is connected so that the user is enabled to wear the pouch with cylindrical tubes in a firm and locked position irrespective of inner wear of the user. In an embodiment, the open end and the closed end of the pouch are separated by a non-return valve positioned in the pouch and after the extension of penis such that the urine that passes from the open end of the pouch to the closed end of the pouch is prevented from flowing back. In an embodiment, a section of the pouch proximal to the closed end of the pouch is defined as a bag for the collection of urine at the closed end, wherein the bag comprises the absorbent unit, and wherein the bag is strapped on an inner thigh of the user.

In an embodiment, the pouch is defined as a membrane that is formed of a latex formulation. In an embodiment, the pouch is lubricated with a high viscosity lubricant to enable smooth insertion of the penis. In an embodiment, the holding device further comprises an air release passage to enable removal of air trapped within the pouch while the user wears the male incontinence device. In another embodiment, the male incontinence device further comprises a band that extends from the cylindrical tube, wherein the band is loops around the base of the scrotum of the user.

In an embodiment, the male incontinence device further comprises of a cylindrical tube with a series of grooves, wherein at the anterior end, adjacent to the base of the penis has a cap (annular locking ring) that locks a thin silicone flexible tube. The silicone flexible tube is kept rolled. At the posterior end of the cylindrical tube, the flexible pouch is unrolled on to the cylindrical tube in such a way that the bead at the open end of the flexible pouch is placed in one of the grooves. In an embodiment, to complete the locking, the silicon flexible tube is unrolled onto and to cover area of the flexible pouch and opens at a posterior end of the cylindrical tube. A sponge ring is placed on an inner surface of the cylindrical tube to be the contact surface with the penis, wherein the sponge ring is locked between a flange at the posterior end of the cylindrical tube and the annular locking ring at the anterior end of the of the cylindrical tube.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and components disclosed herein. The description of a method step or a component referenced by a numeral in a drawing is applicable to the description of that method step or component shown by that same numeral in any subsequent drawing herein.

FIG. 1A shows a schematic drawing of the male incontinence device, as an embodiment of the present invention.

FIG. 1B shows a schematic cross section of the cylindrical tubes of the male incontinence device, as an embodiment of the present invention.

FIG. 1C shows a schematic cross section at the open end with flange of the holding mechanism of the male incontinence device as an embodiment of the present invention.

FIG. 2A shows a front schematic view of the male incontinence device that is inserted over the penis of a user, as a first embodiment of the present invention.

FIG. 2B shows a side sectional schematic view of the male incontinence device that is inserted over the penis of a user, as a first embodiment of the present invention.

FIG. 3A shows a front schematic view of the male incontinence device that is inserted over the penis of a user, as a second embodiment of the present invention.

FIG. 3B shows a side sectional schematic view of the male incontinence device that is inserted over the penis of a user, as a second embodiment of the present invention.

FIG. 4A shows a front schematic view of the male incontinence device that is inserted over the penis of a user, as a third embodiment of the present invention.

FIG. 4B shows a side sectional schematic view of the male incontinence device that is inserted over the penis of a user, as a third embodiment of the present invention.

FIG. 5A shows a front schematic view of the male incontinence device that is inserted over the penis of a user, as a fourth embodiment of the present invention.

FIG. 5B shows a side sectional schematic view of the male incontinence device that is inserted over the penis of a user, as a fourth embodiment of the present invention.

FIG. 6 shows a side sectional view of the male incontinence device that is inserted over the penis of the user, as a fifth embodiment of the present invention.

FIGS. 7A-7C show another embodiment of the construction of the holding device by way of a cylindrical tube with a series of grooves, as a sixth embodiment of the present invention.

### DETAILED DESCRIPTION OF THE DRAWINGS

Exemplary embodiments now will be described. The disclosure may, however, be embodied in many different forms and should not be construed as limited to the embodiments set forth herein; rather, these embodiments are provided so that this disclosure will be thorough and complete, and will fully convey its scope to those skilled in the art. The terminology used in the detailed description of the particular exemplary embodiments illustrated in the accompanying drawings is not intended to be limiting. In the drawings, like numbers refer to like elements.

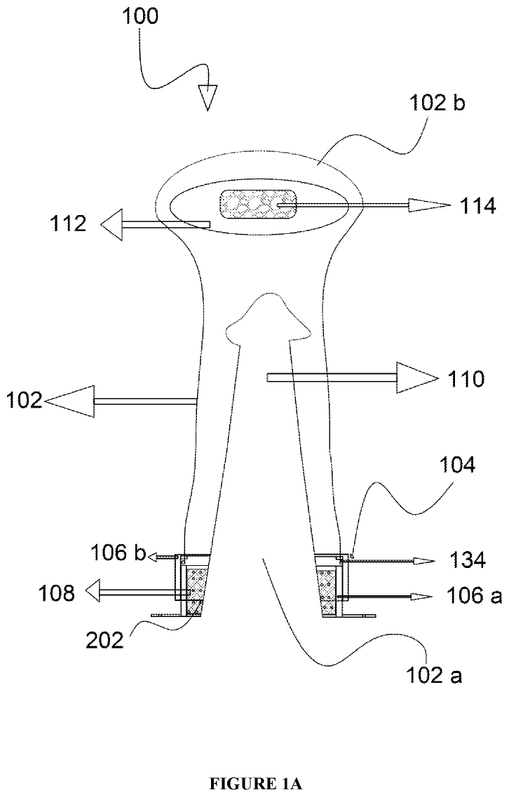

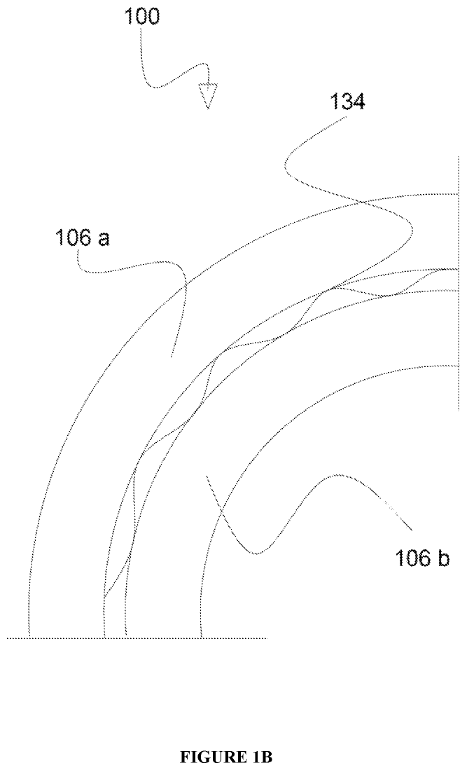

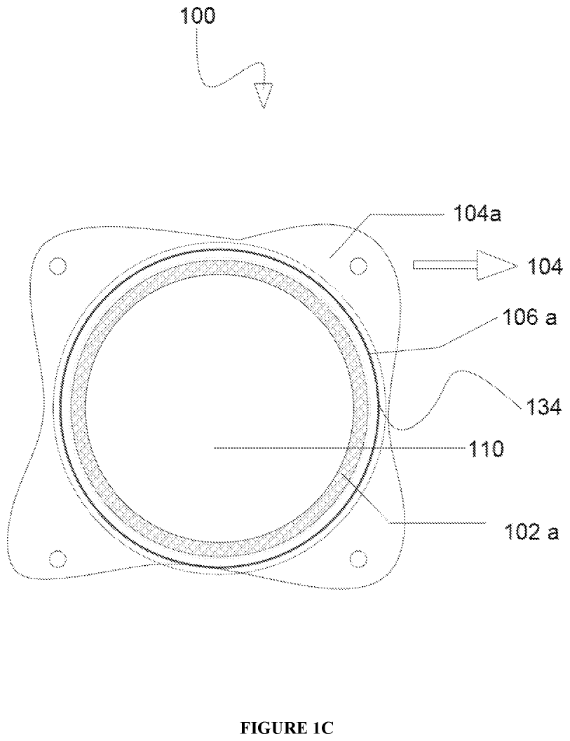

FIG. 1A shows a schematic drawing of the male incontinence device 100, as an embodiment of the present invention. The male incontinence device 100 disclosed here comprises a pouch 102 with an open end 102a and a closed end 102b. The open end 102a has a bead 134 that is locked between the two cylindrical tubes of the holding mechanism 104. The holding mechanism 104 comprises of two cylindrical tubes 106a and 106b and a tubular sponge 108. The two cylindrical tubes 106a and 106b are designed telescopically with a groove or recess to locate the bead 134 of the pouch 102. The tubular sponge 108 forms the inner most layer and is flush with the inner wall of the inner tube 106a. The tubular sponge 108 is in expanded or squeezed position depending on the state of the penis 110, either erect or flaccid. When the penis 110 is flaccid, the tubular sponge 108 is expanded and when penis 110 is erect the tubular sponge 108 is squeezed.

The closed end of the pouch 102 has a flared end that accommodates an absorbent unit 112 consisting of a sponge embedded with absorbent material to collect urine in one of solid and gel form. As used herein, the “absorbent unit” is referred to a sponge material that has liquid absorbing properties, for example, a cellulose or poly urethane sponge. An absorbent powder 114 is positioned inside the absorbent unit 112, for example, sodium polyacrylate, to convert the urine into a solid or gel form. In an embodiment, the pouch 102 is lubricated with high viscosity fluid to enable smooth insertion of the penis 110. In other words, the high viscosity fluid enables the surface tension property of the fluid to adhere the pouch to the penis 110. The high viscosity fluid is not only for ease in insertion, but for holding the pouch 102 in place by the surface tension property of the fluid. An antibacterial agent can also be added to the fluid to prevent any possible rash during extended usage of the male incontinence device 100. The holding device 104 also comprises an air release passage 202 as shown in FIG. 1, to enable removal of air trapped within the pouch 102 while the user wears the male incontinence device 100.

FIG. 1B shows a schematic cross section of the cylindrical tubes 106a and 106b of the male incontinence device 100, as an embodiment of the present invention and FIG. 1C shows a schematic cross section at the open end with flange of the holding mechanism of the male incontinence device 100 as an embodiment of the present invention. Referring to FIGS. 1B and 1C, the two cylindrical tubes 106a and 106b are joined together with the bead 134 in a trapped position in between or in a sandwiched manner with an easy release mechanism to enable the user to slide the cylindrical tubes 106a and 106b away from each other and remove the trapped bead 134. The bead 134 is positioned in a groove at the top end of the holding device 104. Furthermore, the cylindrical tubes 106a comprise a recess in the inner layer, into which the tubular sponge 108 can be placed in a locked position.

The tubular sponge 108 serves the purpose of holding in position the cylindrical tubes 106a and 106b and the connected pouch 102, at the base of the penis 110. The tubular sponge 108 allows accommodation of the varying size of penis 110 to be held in snug fit with the cylindrical tube 106a and 106b in such a manner that the connected pouch 102 covers the penis 110 all the time it is worn by the user. The closed end 102b of the pouch 102 accommodates an absorbent unit 112 containing a polyacrylate based powder or polybeads, a hydrogel forming polymer.

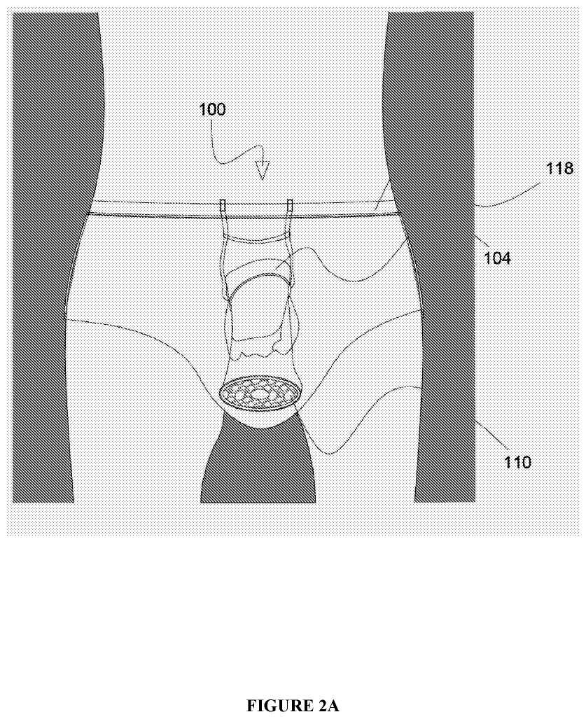

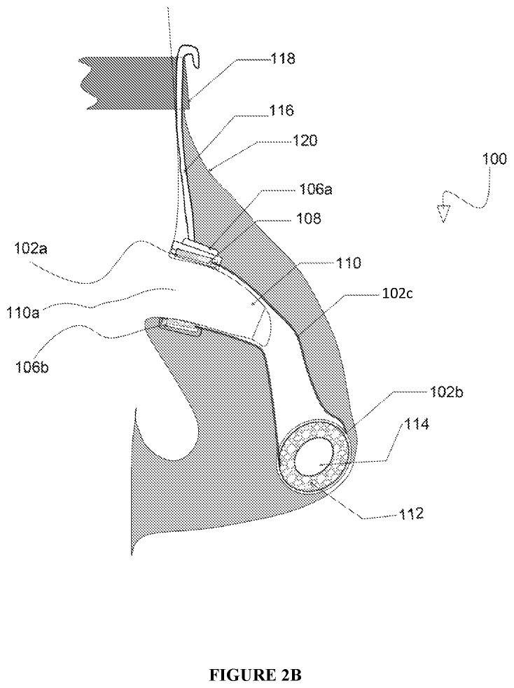

Referring to FIGS. 2A and 2B, FIG. 2A shows a front schematic view of the male incontinence device 100 that is inserted over the penis 110 of a user, as a first embodiment of the present invention. FIG. 2B shows a side sectional schematic view of the male incontinence device 100 that is inserted over the penis 110 of a user, as a first embodiment of the present invention. As shown in FIGS. 2A and 2B, the male incontinence device 100 comprises the pouch 102 with a closed end 102a and an open end 102b. The closed end 102b is flared and comprises a connecting narrow neck 102c of smaller diameter in comparison to the rest of the pouch 102. The open end 102a of the pouch 102 has a bead 134 positioned securely in the holding mechanism 104. The pouch 102, can generally be made of flexible material like natural latex or poly urethane with some elasticity to facilitate a flaccid or turgid state of penis during use. However, the pouch 102 can also be made of a simple polyethylene without elastic properties, if cost of the final product to user becomes an overriding factor. In another embodiment, the pouch 102 is also defined as a membrane that is formed of special latex formulation, but is not limited to a latex formulation and may comprise any other material with similar properties. As shown in FIGS. 1A-1C, the open end 102a of the pouch 102 is connected to two cylindrical tubes 106a and 106b that slide one over another and lock the bead 134 between the inner surface of one tube 106b and outer surface of second tube 106a.

The absorbent unit 112 expands into the flared closed end 102b and is locked into position by a narrow neck 102c connecting the closed flared end 102b with the body of the pouch 102. In an embodiment, the two cylindrical tubes 106a and 106b that are connected to the pouch 102 at the bead 134 are held covering the penis 110 at the base of the penis 110 by two connecting rods 116, which are firm and yet pliable to be shaped accordingly. The connecting rods 116 are connected to the cylindrical tubes 106a and 106b at right angle and extend up to the navel of the user such that the connected rods 116 are held in position by the elastic band 118 in the inner wear 120 of the user.

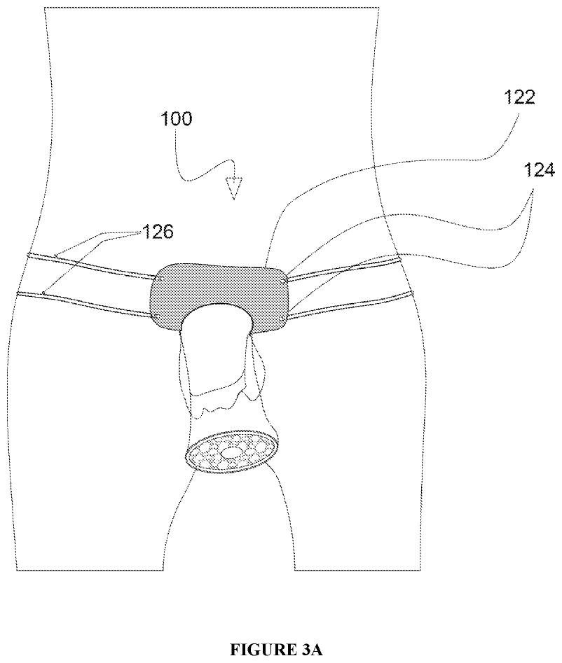

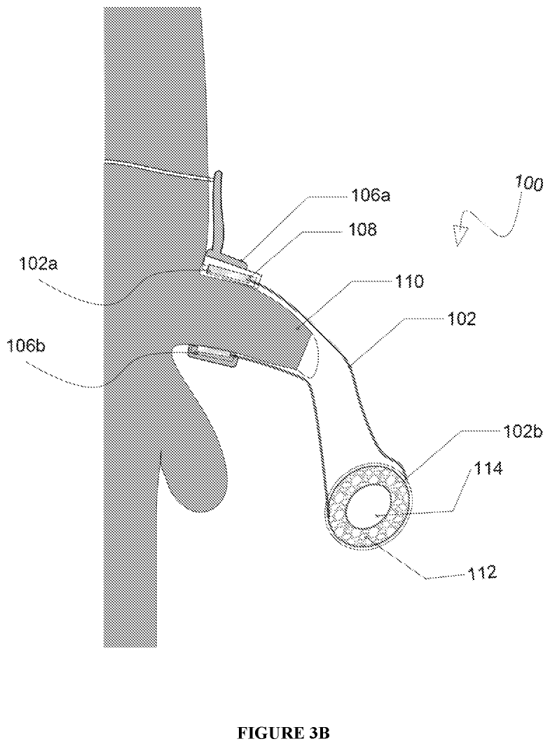

Referring to FIGS. 3A and 3B, FIG. 3A shows a front schematic view of the male incontinence device 100 that is inserted over the penis 110 of a user, as a second embodiment of the present invention. FIG. 3B shows a side sectional schematic view of the male incontinence device 100 that is inserted over the penis 110 of a user, as a second embodiment of the present invention. In this embodiment, the bead 134 of pouch 102 is integrally secured between the cylindrical tubes 106a and 106b in such a manner that the user wears the pouch 102 in a firmly locked position at the base of the penis and in contact with the tubular sponge 108 at the innermost diameter of the cylindrical tubes 106a and 106b, to enable the tubular sponge 108 to expand and contract inversely to the varying diameter of one of the flaccid, turgid penis 110 of user at different times. The cylindrical tubes 106a and 106b comprise a flange 122 that is integrated to the cylindrical tubes 106a and 106b, where the flange 122 comprises anchor holes 124 into which an elastic thread 126 is connected so that the user is enabled to wear the whole contraption of the pouch 102 with cylindrical tubes 106a and 106b in a firm and locked position irrespective of the inner wear 120 of the user.

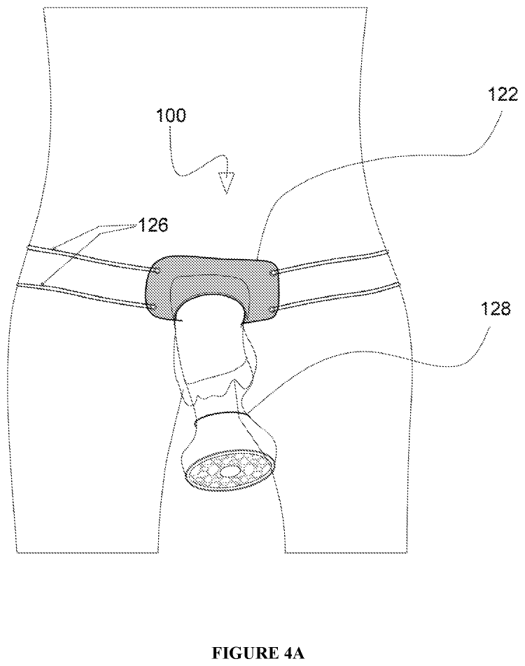

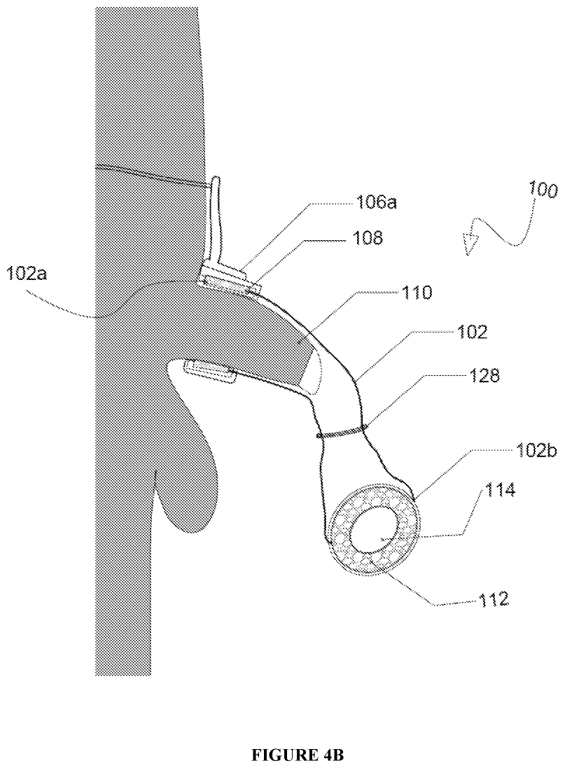

Referring to FIGS. 4A and 4B, FIG. 4A shows a front schematic view of the male incontinence device 100 that is inserted over the penis 110 of a user, as a third embodiment of the present invention. FIG. 4B shows a side sectional schematic view of the male incontinence device 100 that is inserted over the penis 110 of a user, as a third embodiment of the present invention. In this embodiment, the open end 102a is connected to two cylindrical tubes 106a and 106b with the tubular sponge 108 and the closed end 102b comprises the absorbent unit 112 embedded with an absorbent like sodium polyacrylate or such polygel. The open end 102a with the two cylindrical tubes 106a and 106b are connected is the anchor and the closed end 102b with the absorbent unit 112 is for urine collection. These open end 102a and closed end 102b of the pouch 102 are separated by a non-return valve 128 (as shown in FIGS. 4A and 4B) positioned over the pouch 102 after the extension of penis 110 such that the urine that passes from the open end 102a of the pouch 102 to the closed end 102b of the pouch 102 is prevented from flowing back.

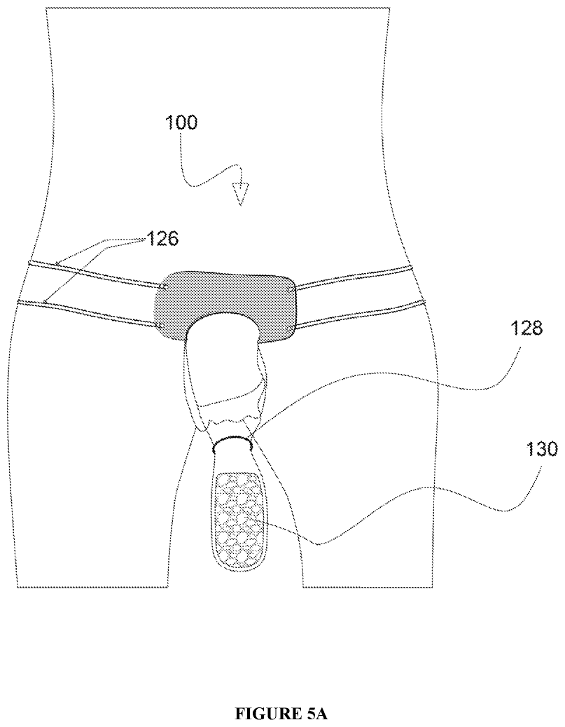

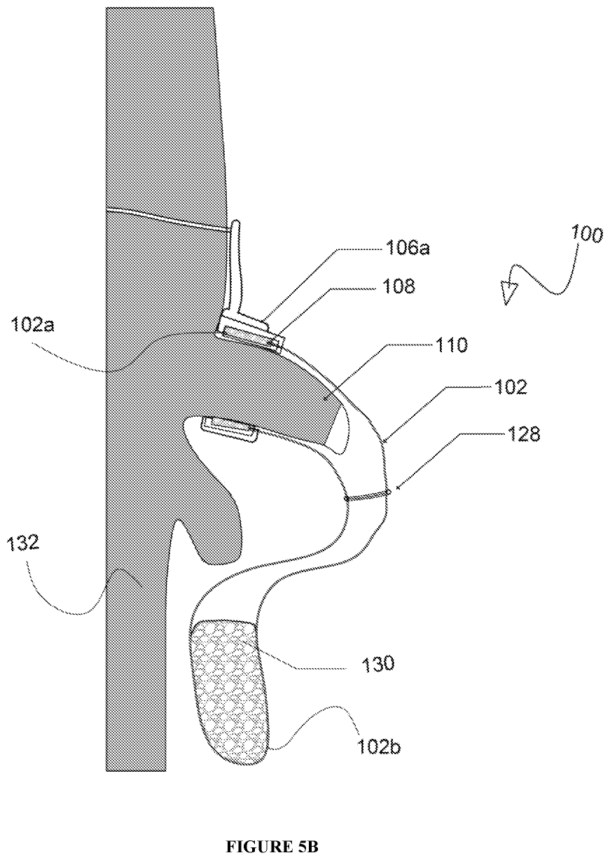

Referring to FIGS. 5A and 5B, FIG. 5A shows a front schematic view of the male incontinence device 100 that is inserted over the penis 110 of a user, as a fourth embodiment of the present invention. FIG. 5B shows a side sectional schematic view of the male incontinence device 100 that is inserted over the penis 110 of a user, as a fourth embodiment of the present invention. In this embodiment, a section of the pouch 102 proximal to the closed end 102b is defined as a bag 130 for the collection of urine at the closed end 102b, wherein the bag 130 comprises the absorbent unit 112 and absorbent polygel. This forms the shape of a bag 130 that is strapped on an inner thigh 132 of the user as shown in FIG. 5B.

In an embodiment, the male incontinence device 100 as shown in FIGS. 5A and 5B also comprises a liquid collection section, for example, in the form of a urine collection vial for collecting approximately 15 ml of urine, before being converted to solid for patients who are hospitalised. This embodiment of the urine collection vial is designed based on requirements in hospitals where a doctor can check the colour of the urine as part of diagnosis and determine the condition of the patient, which may not be possible if the entire urine collected is converted to solid.

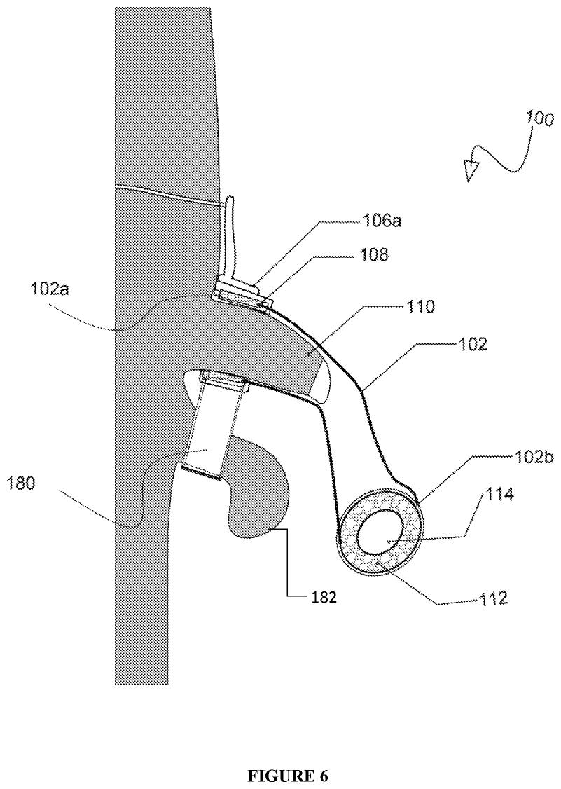

Referring to, FIG. 6 shows a side sectional view of the male incontinence device 100 that is inserted over the penis 110 of the user in accordance with the fifth embodiment of the present invention. As shown in FIGS. 6, a band 180 extends from the cylindrical tube 106a, wherein the band 180 loops around the scrotum 182, thereby anchoring the male incontinence device 100 in a firm position such the pouch covers the penis of the user during usage.

In all the embodiments described before, the tubular sponge 108 besides locking the pouch 102 at the base of the penis 110, also acts as an absorbing unit for any backflow of urine.

In all the embodiments described so far, the closed end of the pouch 102 has polyacrylate based powder or polybeads, a hydrogel forming polymer held in any method other than absorbent unit 112 and still performs the functions of liquid absorption.

In all embodiments described so far, the inner cylindrical tube can also be conceived as two parts, which the user can conjoin to encircle at the base of the penis.

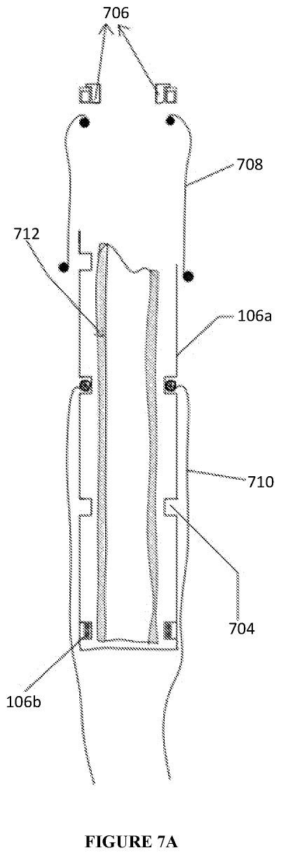

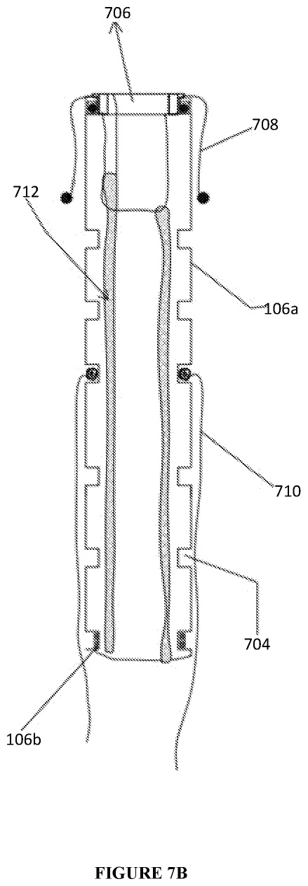

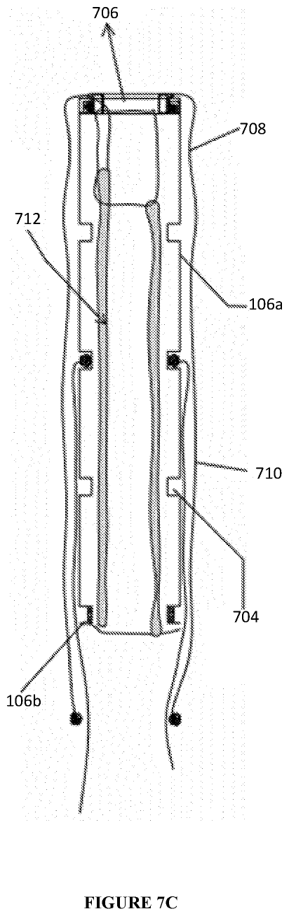

FIGS. 7A-7C show another embodiment of the construction of the holding device 104 is by way of a cylindrical tube 106 with a series of grooves 704, as an embodiment of the present invention. At the anterior end (base of the penis) it has a cap 706 (annular lock ring) which locks a thin flexible tube 708, preferably made of silicone. This silicone flexible tube 708 is kept rolled. At the posterior end, the flexible pouch 710 is unrolled onto the cylindrical tube 106 in such a way that the bead 134 at the open end of the flexible pouch 710 is placed in one of the grooves 704. In order to complete the locking, the silicon flexible tube 708 is unrolled to cover area of the flexible pouch 710 and opens at the posterior end of the cylindrical tube 106. A sponge ring 712 is placed on the inner surface of the cylindrical tube 106 to be the contact surface with the penis 110. The sponge ring 712 is locked between the flange end at the posterior end of cylindrical tube 106 and annular lock ring 706 at the anterior end.

The concept of locking the bead 134 in a groove 704 is achieved by locking with a second thin film. The two thin films one over the other will hug the cylindrical tube 106 with grooves 704, thereby locking the bead 134 of the flexible pouch 710. Alternatively, the bead 134 in the groove 704 can be kept in place by a wide width rubber band or a simple O ring. An adhesive medical plaster can also be also used, which can be removed after use. These are few examples of groove held pouch locking to a holding device. The series of grooves 704 also serve the purpose of multiple resistance points. For example, if the bead 134 slips out of one groove 704 it is locked again in a second groove 704. This way multiple safety points helps to hold the flexible pouch 710 in place in the holding device 104.

While the present disclosure has been described with reference to certain embodiments, it will be understood by those skilled in the art that various changes may be made and equivalents may be substituted without departure from the scope of the present disclosure. In addition, many modifications may be made to adapt a particular situation or material to the teachings of the present disclosure without departure from its scope. Therefore, it is intended that the present disclosure not be limited to the particular embodiment disclosed, but that the present disclosure will include all embodiments that fall within the scope of the appended claims.

I claim:

1. A male incontinence device comprising:
a holding device comprising two cylindrical tubes that locks in an integral manner and a tubular sponge;
a pouch comprising an open end and a closed end and extending from an outside surface of the cylindrical tubes to extend beyond penis that is inserted within, wherein a closed end of the pouch has a flared section that accommodates an absorbent unit which comprises of a sponge embedded with a super absorbent material to absorb urine; and
a bead positioned at the open end of the pouch is locked between the two cylindrical tubes.

2. The male incontinence device as claimed in claim 1, wherein the tubular sponge forms the inner most layer and is flush with the inner wall of the inner tube, and wherein the tubular sponge ensures that no back-flow of urine passes through.

3. The male incontinence device as claimed in claim 1, wherein the tubular sponge is in one of an expanded and squeezed position depending on state of the penis, wherein when the penis is flaccid, the tubular sponge is expanded, and when penis is erect, the tubular sponge is squeezed.

4. The male incontinence device as claimed in claim 1, wherein the cylindrical tubes that are connected to the pouch are held covering the penis at the base of the penis by two connected rods.

5. The male incontinence device as claimed in claim 1, wherein the cylindrical tubes comprise a flange that is integrated to the cylindrical tubes, where the flange comprises anchor holes into which an elastic thread is connected so that the user is enabled to wear the pouch with cylindrical tubes in a firm and locked position irrespective of inner wear of the user.

6. The male incontinence device as claimed in claim 1, wherein the open end and the closed end of the pouch are separated by a non-return valve positioned over the pouch and after the extension of penis such that the urine that passes from the open end of the pouch to the closed end of the pouch is prevented from flowing back.

7. The male incontinence device as claimed in claim 1, wherein a section of the pouch proximal to the closed end of the pouch is defined as a bag for the collection of urine at the closed end, wherein the bag comprises the absorbent unit, and wherein the bag is strapped on an inner thigh of the user.

8. The male incontinence device as claimed in claim 1, wherein the pouch is lubricated with a high viscosity lubricant to enable smooth insertion of the penis.

9. The male incontinence device as claimed in claim 1, wherein the holding device further comprises an air release passage to enable removal of air trapped within the pouch while the user wears the male incontinence device.

10. The male incontinence device as claimed in claim 1, further comprises a band that extends from the cylindrical tube, wherein the band supports scrotum of the user while the user urinates.

11. The male incontinence device as claimed in claim 1, further comprising a cylindrical tube with a series of grooves, wherein an anterior end adjacent to the penis comprises has an annular locking cap that locks a thin silicon flexible tube, wherein the silicon flexible tube is kept rolled and the at the posterior end the flexible pouch, the silicon flexible tube is unrolled on to the cylindrical tube in such a way that the bead at the open end of the flexible pouch is placed in one of the grooves.

12. The male incontinence device as claimed in claim 11, wherein to complete the locking, the silicon flexible tube is unrolled onto to cover area of the flexible pouch and opens at a posterior end of the cylindrical tube, wherein a sponge ring is placed on an inner surface of the cylindrical tube to be the contact surface with the penis, wherein the sponge ring is locked between a flange end at the posterior end of the cylindrical tube and at the anterior end of the cylindrical tube.
